(19) United States
(12) Patent Application Publication
(10) Pub. No.: US 2016/0168531 A1
(43) Pub. Date: Jun. 16, 2016

(54) RECOVERY AND PURITY OF MAGNETICALLY TARGETED CELLS

(71) Applicant: BIOMAGNETIC SOLUTIONS LLC, State College, PA (US)

(72) Inventor: Paul A. Liberti, Naples, FL (US)

(21) Appl. No.: 14/937,855

(22) Filed: Nov. 10, 2015

Related U.S. Application Data
(60) Provisional application No. 62/077,348, filed on Nov. 10, 2014.

(51) Int. Cl. C12M 1/00

(52) U.S. Cl. CPC C12M 47/04

(57) ABSTRACT

Methods and apparatus for separation of rare cells of interest from a biological sample are provided. The present methods and apparatus effectively reduce fragile rare cell exposure to manipulations that adversely impact cell integrity and collection.

This application claims priority to U.S. Provisional Application No. 62/077,348 filed Nov. 10, 2014, the entire disclosure being incorporated herein by reference as though set forth in full.

### FIELD OF THE INVENTION

This invention relates to immuno-magnetic separations, more specifically to the separation of cells that exist in complex mixtures at low frequencies and cell separations in general. Methods and means are disclosed for substantially improving such separations as well as latitude of manipulation that here-to-fore have not been possible. Those latitudes simplify processing and facilitate manual and automated systems.

### BACKGROUND OF THE INVENTION

Numerous publications and patent documents are cited throughout the specification in order to describe the state of the art to which this invention pertains. Each of these citations is incorporated herein by reference, particularly as related to the protocols described therein, as though set forth in full.

There are numerous manufacturing, analytical and laboratory processes and procedures which involve specific binding pair interactions. Many laboratory and clinical procedures are based on such interactions, referred to as bio-specific affinity reactions. Such reactions are commonly used in diagnostic testing of biological samples, or for the separation of a spectrum of target substances, especially biological entities such as cells, viruses, proteins, nucleic acids and the like. It is important in practice to perform the specific binding pair interactions as quickly and efficiently as possible. The effectiveness of such reactions depends on classical chemical variables such as temperature, concentration and binding affinity of pair members for one another. The use of high binding affinity pairs is important, particularly when the concentration of one of the specific binding pair members to be isolated is in extremely low concentrations, as often is the case in biological systems

Various methods are available for binding, separating or analyzing the target substances mentioned above based upon complex formation between the substance of interest and another substance to which the target substance specifically binds. Separation of the resulting complexes from solution or from unbound material may be accomplished gravitationally, e.g. by settling, or, alternatively, by centrifugation of finely divided particles or beads coupled to the ligand substance. If desired, such particles or beads may be made magnetic to facilitate the bound/free separation step.

Magnetic particles are well known in the art, as is their use in immune and other bio-specific affinity reactions. See, for example, U.S. Pat. No. 4,554,088 and Immunoassays for Clinical Chemistry, pp. 147-162, Hunter et al. eds., Churchill Livingston, Edinborough (1983). Generally, any material which facilitates magnetic or gravitational separation may be employed for this purpose. However, processes relying on magnetic principles are preferred.

Magnetic particles generally fall into two broad categories. The first category includes particles that are permanently magnetizable, or ferromagnetic; and the second comprises particles that demonstrate bulk magnetic behavior only when subjected to a magnetic field. The latter are referred to as magnetically responsive particles. Materials displaying magnetically responsive behavior are sometimes described as superparamagnetic. However, materials exhibiting bulk ferromagnetic properties, e.g., magnetic iron oxide may be characterized as superparamagnetic when provided in crystals of about 30 nm or less in diameter. Larger crystals of ferromagnetic materials, by contrast, retain permanent magnet characteristics after exposure to a magnetic field and tend to aggregate thereafter due to strong particle-particle interaction.

Magnetic particles can be classified as large (1.5 to about 50 microns), small (0.7-1.5 microns), and colloidal or nanoparticles (<200 nm). The latter are also called ferrofluids or ferrofluid-like particles and have many of the properties of classical ferrofluids. Liberti et al pp 777-790, E. Pelezzetti (ed) “Fine Particle Science and Technology, Kluver Acad. Publishers, Netherlands, Small magnetic particles are quite useful in analyses involving bio-specific affinity reactions, as they are conveniently coated with bio-functional polymers (e.g., proteins), provide very high surface areas and give reasonable reaction kinetics. Magnetic particles ranging from 0.7-1.5 microns have been described in the patent literature, including, by way of example, U.S. Pat. Nos. 3,970,518; 4,018,886; 4,230,685; 4,267,234; 4,452,773; 4,554,088; and 4,659,678. Certain of these particles are disclosed to be useful solid supports for immunologic reagents. In addition to the small magnetic particles mentioned above, there is a class of large magnetic particles (>1.5microns to about 50 microns) which also have superparamagnetic behavior. Such materials include those invented by Ugelstad (U.S. Pat. No. 4,654,267) and manufactured by Dynal, (Oslo, Norway, now available through Invitrogen-Life Technologies). Polymer particles are synthesized, and through a process of particle swelling, magnetite crystals are embedded therein. Other materials in the same size range are prepared by performing the synthesis of the particle in the presence of dispersed magnetite crystals. This results in the trapping of magnetite crystals thus making the materials magnetic. In both cases, the resultant particles have superparamagnetic behavior, readily dispersing upon removal of the magnetic field. Unlike magnetic colloids or nano-particles referred to above, such materials, as well as small magnetic particles, because of the mass of magnetic material per particle are readily separated with simple laboratory magnetics. Thus, separations are effected in gradients as low as a few hundred gauss/cm to about 1.5 kilogauss/cm.

Based on theoretical calculations, colloidal magnetic particles (below approximately 200 nm) require substantially higher magnetic gradients—on the order of 100 kGauss/cm—for separation because of their diffusion energy, small magnetic mass/particle ratio and stoke drag. In spite of that Liberti (unpublished results) discovered they can be separated in fields as low as 7-10 kGauss/cm. Dr. Liberti theorized such materials must be forming nano-particle magnetic chains' in magnetic fields resulting in a dramatic alteration in mass thereby confounding theoretical calculations.

U.S. Pat. No. 4,795,698 to Owen et al. relates to polymer coated, sub-micron size colloidal superparamagnetic particles. The '698 patent describes the manufacture of such particles by precipitation of a magnetic species in the presence of a bio-functional polymer. The structure of the resulting particles, referred to herein as single-shot particles, has been found to be a micro-agglomerate in which one or more ferromagnetic crystallites having a diameter of 5-10 nm are embedded within a polymer body having a diameter on the order of 50 nm. These particles exhibit an appreciable tendency not to separate from aqueous suspensions for observation periods as long as several months. Molday (U.S. Pat. No. 4,452,773) describe a material which is similar in properties to those described in the '698 patent of Owen et al. produced by forming magnetite and other iron oxides from Fe⁺³/Fe⁺² via base addition in the presence of very high concentrations of dextran. Materials produced in this manner have colloidal properties. This process has been commercialized by Miltenyi Biotec, (Bergisch Gladbach, Germany). Those products have proved to be very useful in cell separation assays.

Another method for producing superparamagnetic colloidal particles is described in U.S. Pat. No. 5,597,531 and U.S. Pat. No. 6,551,843. In contrast to the particles described in the '698 patent, these latter particles are produced by directly coating a bio-functional polymer onto pre-formed super-paramagnetic crystals which have been dispersed by sonic energy into quasi-stable crystalline clusters ranging from about 25 to 120 nm. The resulting particles, referred to herein as direct-coated or DC particles, exhibit a significantly larger magnetic moment than the nanoparticles of Owen et al. or Molday et al. having the same overall size. Magnetic separation techniques utilize magnetic field generating apparatus to separate ferromagnetic bodies from the fluid medium. In contrast, the tendency of colloidal superparamagnetic particles to remain in suspension, in conjunction with their relatively weak magnetic responsiveness, requires the use of high-gradient magnetic separation (HGMS) techniques in order to separate such particles from a fluid medium in which they are suspended. In HGMS systems, the gradient of the magnetic field, i.e., the spatial derivative, exerts a greater influence upon the behavior of the suspended particles than is exerted by the strength of the field at a given point. High gradient magnetic separation is useful for separating a wide variety of biological materials, including eukaryotic and prokaryotic cells, viruses, nucleic acids, proteins, and carbohydrates. In methods known heretofore, biological material has been separable by means of HGMS if it possesses at least one characteristic determinant capable of being specifically recognized by and bound to a binding agent, such as an antibody, antibody fragment, specific binding protein (e.g., protein A, streptavidin), lectin, and the like.

HGMS systems can be divided into two broad categories. One such category includes magnetic separation systems that employ a magnetic circuit that is situated externally to a separation chamber or vessel wherein the magnetic gradient is created by pole piece placement and design. Examples of such external separators (or open field gradient separators) are described in U.S. Pat. No. 5,186,827. In several of the embodiments described in the '827 patent, the requisite magnetic field gradient is produced by positioning permanent magnets around the periphery of a non-magnetic container such that the like poles of the magnets are in a field-opposing configuration. The extent of the magnetic field gradient within the test medium that may be obtained in such a system is limited by the strength of the magnets and the separation distance between the magnets. Hence, there is a finite limit to gradients that can be obtained with an external gradient system.

Another type of HGMS separator utilizes a ferromagnetic collection structure that is disposed within the test medium in order to 1) intensify an applied magnetic field and 2) produce a magnetic field gradient within the test medium. In one known type of internal HGMS system, fine steel wool or gauze is packed within a column that is situated adjacent to a magnet or within a magnetic dipole. The applied magnetic field by well-known principles of physics results in the creation of very high gradients extending from the surfaces of the wires so that suspended magnetic particles will be attracted toward, and adhere to, the surfaces of the wires. The gradient produced on such wires is inversely proportional to the wire diameter whereas magnetic “reach” decreases with diameter. Hence, very high gradients can be generated. One drawback of internal gradient systems is that the use of steel wool, gauze material, or steel microbeads, may entrap non-magnetic components of the test medium by capillary action in the vicinity of intersecting wires or within interstices between intersecting wires. Various coating procedures have been applied to such internal gradient columns (U.S. Pat. Nos. 4,375,407 & 5,693,539), however, the large surface area in such systems still creates recovery issues via adsorption. Hence, internal gradient systems are not desirable where recovery of very low frequency captured entities is the goal of the separation. Further, they make automation difficult and costly.

On the other hand, cell separations using HGMS based approaches with external gradients provide a number of conveniences. First, simple laboratory tubes such as test tubes, centrifuge tubes, or even vacutainers (used for blood collection) may be employed. When external gradients are of the kind where separated cells can, in principle, effectively be monolayered, as is the case with appropriately designed quadrupole/hexapole devices as described in U.S. Pat. No. 5,186,827 or the opposing dipole arrangement described in U.S. Pat. No. 5,466,574, washing of cells and subsequent manipulations are facilitated. Furthermore, recovery of the cells from tubes or similar containers is a simple and efficient process. This is particularly the case when compared to recoveries from high gradient columns. Such separation vessels also provide another important feature: the ability to reduce the volume of the original sample. For example, if a particular human blood cell subset, (e.g. magnetically labeled CD34+ cells), is isolated from blood diluted 20% with buffer to reduce viscosity, a 15 ml conical test tube may be employed as the separation vessel in an appropriate quadrupole magnetic device.

After appropriate washes and/or separations and resuspensions to remove non-bound cells, CD34+cells can very effectively be resuspended in a volume of 200 μl. This can be accomplished, for example, by starting with 12 ml of solution (blood, ferrofluid and dilution buffer) in a 15 ml conical test tube, performing a separation, discarding the supernatant and subsequent wash supernatants and resuspending the recovered cells in 3 ml of appropriate cell buffer. A second separation is then performed which may include additional separation/wash steps (as might be necessary for doing labeling or staining reactions) and finally the isolated cells are easily resuspended in a final volume of 200 μl. By reducing volume in this sequential fashion and employing a vortex mixer for resuspension, cells adhered to the tube above the resuspension volumes are recovered into the reduced volume. When done carefully and rapidly in appropriately treated vessels, cell recovery is generally in the range of 60-85% which is some instances is quite suitable. In other applications, e.g., in the case of Circulating Tumor Cells [CTC] or other rare cells in blood, this level of recovery may be unacceptable. Furthermore, it has become abundantly clear to those knowledgeable in CTC isolation that such cells are frail and often in some stage of apoptosis. Accordingly, vortexing and resuspension as well as methodologies that are multistep and prolong the isolation process may well be deleterious to the final CTC yield.

The efficiency with which magnetic separations can be done and the recovery and purity of magnetically labeled cells will depend on many factors. These include such considerations as: the number of cells being separated, the density of targeted determinants present on such cells, the magnetic load per cell, the non-specific binding of the magnetic material (NSB), the technique employed, the nature of the vessel, the composition of the vessel surface, and viscosity of the medium. If non-specific binding [NSB] of a system is relatively constant, as is usually the case, then as the target population decreases so does the purity. For example, a system with 0.2% NSB that recovers 80% of a population which is 0.20% in the original mixture will have a purity of 50%. If, on the other hand, if the initial population was at 1.0%, the purity of the recovered fraction would be 80%.

It is important to note that the smaller the population of targeted cells, the more difficult it will be to magnetically label and recover such cells due to reaction kinetics. Furthermore, labeling and recovery are markedly dependent on the nature of the magnetic particle employed. As an example, large magnetic particles, such as Dynal beads, are too large to diffuse and effectively label cells in suspension through collisions created by mixing of the system. If a cell is in a population of 1 cell per ml of blood, or even less, as could be the case for tumor cells in very early cancers, then the probability of labeling target cells will be related to the number of magnetic particles added to the system and the length of time of mixing. Since mixing of cells with such particles for substantial periods of time will be deleterious, it becomes necessary to increase particle concentration as much as possible. There is, however, a limit to the quantity of magnetic particles that can be added. Instead of dealing with a rare cell mixed in with other blood components, one contends with a rare cell mixed in with large quantities of magnetic particles upon separation. The latter condition does not markedly improve the ability to enumerate such cells or examine them. Hence, the compromise is to limit the quantity of magnetic material and the mixing times, while enabling isolation of very rare target entities.

Another drawback to the use of large particles to isolate cells in rare frequencies (1 to 25-50 per ml of blood) is that large particles tend to cluster around cells in a cage-like fashion making them difficult to “see” or to analyze. Hence, the particles must be released before analysis, which clearly introduces other complications.

In theory, the use of colloidal magnetic particles in conjunction with high gradient magnetic separation appears to be the method of choice for separating a cell subset of interest from a mixed population of eukaryotic cells, particularly if the subset of interest comprises a small fraction of the entire population. With appropriate magnetic loading, sufficient force is exerted on a cell such that it could be isolated even in a media as viscous as that of moderately diluted whole blood. As noted, colloidal magnetic materials below about 200 nm will exhibit Brownian motion that markedly enhances their ability to collide with and magnetically label rare cells. This is well demonstrated, for example, in U.S. Pat. No. 5,541,072. As shown there, very efficient tumor cell purging is obtained employing 100 nm colloidal magnetic particles (ferrofluids). Just as important, colloidal materials at or below the size range noted do not generally interfere with viewing of cells. Cells so retrieved can be examined by flow cytometry or by microscopy employing visible or fluorescent techniques. Because of their diffusive properties, such materials, in contrast to large magnetic particles, readily “find” and magnetically label rare events such as tumor cells in blood. There is, however, a significant problem associated with the use of several ferrofluid-like particle preparations for cell separation in external field gradient systems which, for reasons given above, are the device designs of choice. Direct monoclonal antibody conjugates to sub-micron size magnetic particles of the type described by Owen or Molday, such as those produced by Miltenyi Biotec, do not have sufficient magnetic moment for use in cell selection methods employing the best available external magnetic gradient devices, such as the quadrupole or hexapole magnetic devices described in U.S. Pat. No. 5,186,827. When used for separations in moderately diluted whole blood, they are even less effective. Using similar materials which are substantially more magnetic, such as those described in U.S. Pat. No. 5,597,531 to Liberti and Pino, more promising results have been obtained. In model spiking experiments, it has been found that SKBR3 cells (breast tumor line), while exhibiting a relatively high epithelial cell adhesion molecule (EpCAM) determinant density, are efficiently separated from whole blood with direct conjugates of anti-EpCAM ferrofluids even at very low spiking densities (1-5 cells per ml of blood). On the other hand, PC3 cells (a prostate line) which have low EpCAM determinant density are separated with significantly lowered efficiency. Most likely this is a consequence of inadequate magnetic loading onto these low determinant density cells as well as limitations in magnetic field gradient.

While it is clear that immuno-magnetic separations of targeted cells been extraordinarily useful and have had a huge impact on our knowledge of important biological systems and in their ever increasing importance in cell therapy and cell diagnostics, there is need for improvements in basic methodologies particularly in the case where colloidal magnetic materials are used in conjunction with open field magnetic gradient arrangements. The reasons for this are based on the fact that open field magnetic gradients are limited in the pulling force that can be applied and labeling with colloidal magnetic materials is limited by the amount of magnetic mass that can be placed on a cell surface or any other similar entity. Additionally, cells that might be insufficiently labeled magnetically might be washed away or lost in any of the post magnetic collection steps. In the case of large highly magnetic particles such as Dynabeads, those are generally not issues.

To achieve sufficient magnetic labeling on cells when using colloidal magnetic nano-particles with open field gradients a few rather ingenious methods have been employed. U.S. Pat. No. 6,620,627 describes a method, referred to as controlled aggregation, for loading more magnetic mass onto CTC by first incubating blood suspected of containing CTC with ferrofluid conjugated with anti-EPCAM antibodies. After an appropriate time interval, an agent is added to cause ferrofluid that is free in solution to bind to ferrofluid that is cell bound; thus loading more ferrofluid onto cell surfaces making them more magnetic and easier to separate in the open field gradient devices used. Once collected an agent that reverses ferrofluid aggregation is added thus yielding free ferrofluid and cells that only have ferrofluid bound via the initial immunochemical reaction and therefore cells that are easily interrogated. U.S. Pat. No. 6,551,843B describes another method for increasing magnetic labeling in a rare cell application where determinant density might be limited or where magnetic labeling is not sufficient to yield adequate separation and possibly retention. In the '843 patent, the inventors noted that cells incubated with specific ferrofluid require about 15 minutes to separate, yet when such cells are resuspended and separated a second time, the separation time required is substantially reduced to about 1-2 minutes. From a series of experiments, they concluded that when cells and ferrofluid are moved relative to each other the collision rate increases significantly. Thus they devised a method of sweeping ferrofluid through the cell suspension which results in significantly increased magnetic loading onto cells.

However ingenious such methods as controlled aggregation and sweeping magnetic colloids passed cells in some oscillating fashion might be, it should be asked if such methods are deleterious to cells and are not there better ways to ensure separation and collection of cells that are either difficult to label or that have limited determinants for labeling. In the case of CTC isolation/enrichment, it is well known that such cells are frail and substantial percentages of them are apoptotic. Accordingly, each step in their isolation needs to be done in a fashion that does not destroy or cause fragmentation of cells. Further, wash and resuspension steps can also lead to cell destruction and also cell losses. Better means to accomplish the same ends are clearly needed. Thirdly, for CTC and many other cells where specific cellular determinants are limited in number, there is a need to improve separation efficiency of such cells by means other than magnetic loading. Fourthly, even though tubes and vessels used with open field separators can be used to recover cells in small volumes as described above, the numbers of manipulations required are significant. Given the likelihood that each manipulation can cause loss of, or can negatively impact cell integrity, there is a need to find alternative means to accomplish the same end.

### SUMMARY OF THE INVENTION

In accordance with the present invention, methods and apparatus for improved recovery and purity of magnetically targeted cells of interest are disclosed. The invention provides methods for presenting cells for analysis that do not require transfers from separation container to some observation platform as well as methods for recovering cells in small volumes.

In addition to disclosing means and methods for producing high purity cell subpopulations, we disclose here methods of preserving fragile cells such as circulating tumor cells [CTC] as well as a scheme for their isolation. It has become well known that CTC are more fragile than non malignant cells and in many cases are undergoing apoptosis. (See, for example Qin et al (http://www.cancerci.com/content/14/1/23). Current approaches to sample preparation such as that recommended for CellSearch analysis employ CellSave collection tubes (Streck, Omaha, Neb.) which in addition to preventing coagulation of blood contain components that slowly release formaldehyde which results in fixation of cells and thus preserving cell integrity. Such tubes are widely used for preserving blood cells for flow-cytometry. Recently, Qin et al (http://www.cancerci.com/content/14/1/23) demonstrated that Streck DNA BCT tubes that are designed to prevent cells from ‘leaking’ DNA are as effective as CellSave tubes in preserving cell markers, as determined by flow analysis. They showed that nearly identical results can be obtained on day 4 as on day 1 for blood treated with either of the Streck tubes. In their studies presumably normal blood was spiked with cells from a breast cancer cell line, MCF-7. In spite of their results, a key question is: would the same results be obtained on samples drawn from cancer patients known to have CTC? The latter cells would likely be in various stages of apoptosis and have various immune components on their surfaces. In preliminary experiments done in our lab, in view of results from colleagues at Veridex (Huntingdon Valley, Pa.), it appears that CTC are readily lost by simple sample manipulations, including by centrifugal, washing, and other forces CTC are exposed to in the preparation of a buffy coat or other density-based methods for obtaining leukocytes.

Based on the foregoing we disclose an approach for treating patient blood prior to analysis which can minimize CTC losses that occurs by sheer and pressure forces as well as metabolic and immunological processes. This is procedurally done by collecting patient blood using an as large as possible gauge needle so as to minimize sheering forces that might be deleterious to CTC, discarding the initial part of the draw so as to avoid collecting epithelial cells from the puncture wound and collecting blood into vacutainers that contains not only appropriate anticoagulant(s) but also a variety of inhibitors. Additionally, it would be highly desirable to collect blood samples into pre-chilled vacutainers and thereafter maintain samples, ideally at 4° C. and kept at that temperature to the time of separation/enrichment of CTC. After the blood draw, samples would in rapid succession be centrifuged at relatively low g force sufficient for performing plasma removal as well as platelets that would remain in the plasma fraction when an appropriate g force and time of centrifugation is employed. The inhibitors can be placed in the vacutainers used for collection or introduced to the cells after the plasma-platelet removal step. The following is a listing of inhibitors that should prove beneficial: a. Na Azide at a concentration 0.1-0.3 mM which is well known to shut down metabolic processes (See for example, Palmieri and Klingenberg (European J. Biochem. 1 (1967) 439-446), b. inhibitors of apoptosis such as of c-Myc, Bax, p53, tBid, and BCL that mediate apoptosis as well as caspase inhibitors and other enzymes involved in the apoptotic pathways (all of which that are available at Sigma-Aldrich and other suppliers), c. inhibitors of C1q that will prevent its binding to Ig complexes and also reverse such reaction to some extent. 2, 4 di-amino toluene will suffice for this task as well as others that are well known (Sledge and Bing (J. Biol. Chem. 1973, 248:2818-2823), d. heat aggregated IgG that will bind to C1q in the sample and to Fc cell receptors and e. in the event that one or more biotinylated monoclonal antibodies targeted to CTC is added with the foregoing, then it would be important to add non-specific antibodies in substantial excess that are derived from that species that produced the monoclonals so as to allow the labelling monoclonals to reach their target.

### BRIEF DESCRIPTION OF THE DRAWINGS

FIG. 1 provides a schematic depiction of the mechanism for magnetically separating targeted cells onto a surface, to which has been coupled a component that the magnetic entity can attach to. In addition to disclosing means and methods for producing high purity cell subpopulations, we disclose here methods of preserving fragile cells such as circulating tumor cells [CTC] as well as improved protocols and apparatus for their isolation. It has become well known that CTC are frail and in many cases are undergoing apoptosis. (See, for example Qin et al, on the world wide web at cancerci.com/content/14/1/23. Current approaches to sample preparation such as those recommended for CellSearch analysis employ CellSave collection tubes (Streck, Omaha, Neb.) which, in addition to preventing coagulation of blood contain components that slowly release formaldehyde which results in fixation of cells, thereby preserving cell integrity. Such tubes are widely used for preserving blood cells for flow-cytometry. Recently, Qin et al demonstrated that Streck DNA BCT tubes that are designed to prevent cells from ‘leaking’ DNA are as effective as CellSave tubes in preserving cell markers, as determined by flow analysis. They showed that nearly identical results can be obtained on day 4 as on day 1 for blood treated with either of the Streck tubes. In their studies presumably normal blood was spiked with cells from a breast cancer cell line, MCF-7. In spite of their results, a key question is: would the same results be obtained on samples drawn from cancer patients known to have CTC? Based on the foregoing we disclose an approach for treating patient blood prior to analysis which can minimize CTC losses that occurs by sheer and pressure forces as well as metabolic and immunological processes.

FIG. 2A-FIG. 2B depicts vessels. FIG. 2A shows a vessel that is tube shaped wherein is placed a post thus creating an annular space in which mixtures to be magnetically separated can be placed. In FIG. 2B a vessel fitted with a funnel like top is depicted as well a central post as in FIG. 2A but where in are channel(s) that can be used to fill or evacuate the annular space. The funnel like top can also facilitate filling that space.

FIG. 3A-FIG. 3C show a specially designed separation vessel and its component parts. The vessel is designed to place magnetically targeted entities in regions of highest gradient and to also immobilize targets onto a removable collection surface such as a glass slide. FIG. 3A depicts a ¾ view of the separation vessel. FIG. 3B and FIG. 3C depict a cross section of the separation vessel.

FIG. 4 depicts how four such vessels of FIG. 3 can be fitted into a single quadrupole separator.

FIG. 5A-FIG. 5B depict a cross section of a north-south rectangular magnet array that creates gradient forces such that lines of magnetic entities are collected on a surface that is placed on top of such an array. FIG. 5B depicts cells collected in lines on a surface due to the gradient lines of FIG. 5A.

### DETAILED DESCRIPTION OF THE INVENTION

Cells and/or other magnetically labeled entities of interest must often be separated from solution, without loss or damage to the caused by the processing steps employed to remove unwanted contaminants. Cells must be recovered in an efficient manner and in small volumes or retained in some way that they can easily be interrogated or analyzed. Herein, we disclose means for accomplishing these objectives. The basic concept can be applied to what are referred to as direct or indirect separations wherein a direct separation is done with a ferrofluid that has conjugated to it, for example, a specific Mab. In that case, ferrofluid is directly added to a mixture to be separated, the ferrofluid binds to the entity for which it has specificity and next the entity is separated by applying a magnetic gradient. In the case of the indirect method, typically a Mab, or a Mab conjugated with some unique component that can subsequently be targeted, is added to the mixture, the Mab finds it target, generally excess Mab is removed and that step is followed by adding a ‘common capture’ magnetic material such as a goat α Mouse ferrofluid or where the Mab is biotin labelled and the Streptavidin is conjugated to a ferrofluid.

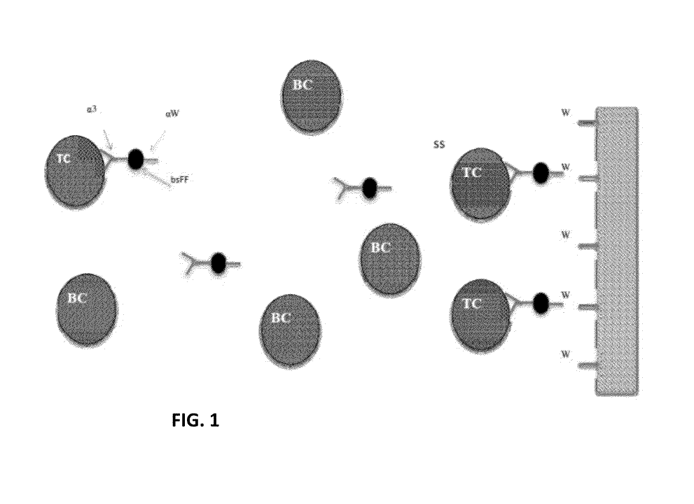

In the case of a direct separation, if a ferrofluid or some other suitable magnetic material has conjugated to it members of at least two binding pairs, this dual specificity can be used advantageously to bind a target entity to some other entity. For example, if a ferrofluid, that has conjugated to it an anti-human CD3 and an anti-W, is mixed with a suspension of human leukocytes, then ferrofluid nano-particles will under appropriate conditions link human CD3 positive cells to component W. Similarly, if W is some component attached to the collection wall of some vessel, which could be the surface of a glass slide that serves as a vessel wall, in time cells will gravitationally settle onto the slide and those cells that are labeled with CD3 will also attach themselves strongly via the reaction of the ferrofluid anti W reaction with the W on the slide. FIG. 1 illustrates this concept where targeted cells are bound to a wall non-covalently by this mechanism. In the illustration, target cells and bystander cells are labeled TC and BC, respectfully, bi-specific ferrofluid is labelled as bsFF and the two such labels on bsFF are αCD3 denoted as α3, that component on the ferrofluid that binds it to the wall or some solid support SS is labelled αW and that component attached to the wall for completing the non-covalent reaction is labelled W. In this illustration, the ferrofluid or nano-particles need not be magnetic, it just needs to be some appropriate bi-specific entity, in which case a ‘common capture’ panning system could be created. In the case where the nano-particles are magnetic and where cells are placed in a vessel where W is conjugated to the inner walls of that vessel labeled SS and where a magnetic gradient is used to bring cells onto the wall, targeted cells would be ‘caught’ on the wall and bystander cells would remain in solution. In that case, contaminating cells that might have placed on or bound to the wall non-specifically [NSB]will be held there by forces less strong than those ‘caught’ by the W-anti W reaction, creating a situation where such contaminants can be more readily removed. Note that this process immediately removes at least one, if not more, processing steps, i.e. the typical resuspension and re-separations. For frail or apoptotic cells, it is very likely that the elimination of one or more manipulative steps is significant. For cells that are only weakly labelled magnetically, catching them on a wall by some strong non-covalent interactions can in principle eliminate loss of cells that might get washed away or even lost by the surface tension created when a vessel is evacuated using a manual or motorized vacuum system, or even in the case where the vessel is evacuated by pouring out the contents. Such a system should significantly improve negative depletions where there is always danger of not depleting loosely held unwanted cells.

For the above general example, in one approach, cells would magnetically be deposited onto a surface of some appropriate vessel where that surface is a microscope slide or some similar device. In that case, the slide portion after appropriate washings and subsequent treatments related to interrogation of attached cells could be directly interrogated by a variety of means well known to those skilled in the art. In this manner, no loss of targeted cells occurs by transfer from collecting vessel to interrogation platform, because there is no transfer. Alternatively, cells could simply be released by simply reversing the W-anti W reaction, where W can be selected from a vast library of member pair reactions that need not be antibody based. Means for doing that are enumerated below.

In conjunction with what has been disclosed and is hereafter referred to as ‘Catch’ or ‘Catch and Release’ [COCAR], we also disclose specially designed companion vessels for performing these processes in a manner that takes greater advantage of the magnetic properties of various open field magnetic arrangements that can be used. For example, it is customary to perform cell and other entity separations using ferrofluid-like nanoparticles in radially symmetric tubes that are subsequently placed in multipole separators, e.g. quadrupole, hexapole, etc., all of the former which are typically designed to produce radial magnetic gradient. However, it is well known that such devices have a region of effectively zero gradients near their centers. And in fact, work reported in 1995 from a joint project between Immunicon Corporation and Systemic/Novartis showed that for a flow device more efficient separation could be obtained if the flow chamber had within it a cylindrical plug placed along the axis of the chamber that would create an annular space such that material to be separated flows around the plug and in close proximity to the magnetic pole pieces where the gradients are highest. J. Ying et al [Biotech and Bioeng, 96 (2007M. Nakamura et al Biotechnol Prog 17 1145-1155 (2001)] confirm those results.

In the case of a static separation employing a vessel that could be placed into a complementary radial gradient maker, such as a test tube within a quadrupole, a central plug placed within that tube clearly eliminates the region of zero gradients and places the material to be separated to be within the high gradient region. Additionally and very importantly, the plug displaces sample such that for a given volume of sample the column height of the sample can be made significantly greater thus creating more surface for monolayer cells or other collected entities. Furthermore, the central plug, which depending on the application need not be on center, could be designed such that with appropriate channeling and valves it can be used for introducing sample, removing sample, adding or removing wash reagents and even the addition and removal of components for performing various reactions on entities so immobilized as described above. Clearly, the same concept can be applied to components that are just held on vessel surfaces via magnetic forces. The added advantage of the Catch or Catch and Release system is that such a vessel can be removed from its magnetic nest once the separation and catch steps have been done.

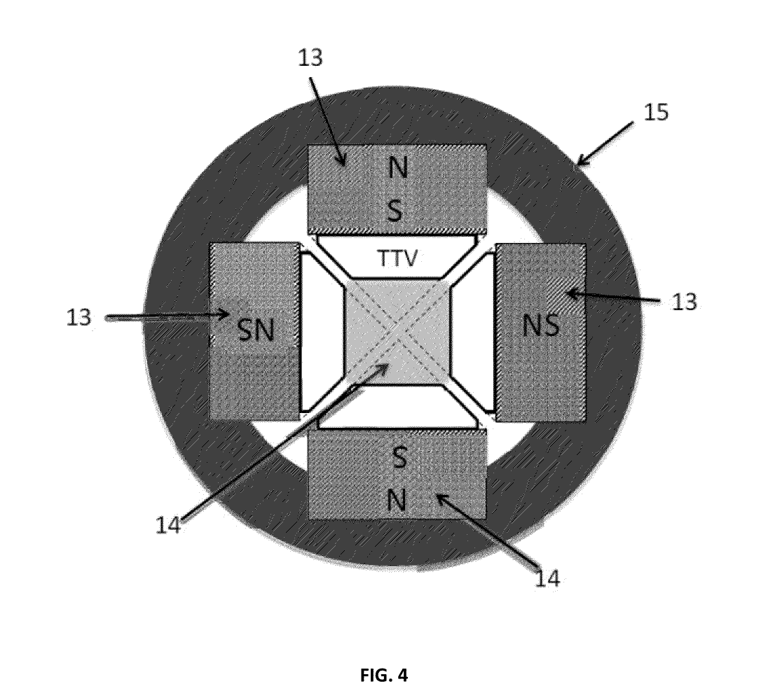

We also disclose other vessels for performing COCAR which is not radially symmetric tubes but instead are constructed such that the surface for collection of a vessel is placed near the face of one magnet within a multipole device such as a quadrupole, hexapole, etc., and the vessel is truncated so as to avoid mixture to be separated from being near the center of quadrupoles thus avoiding regions of low or zero magnetic gradients. In one embodiment a quadrupole could accommodate 4 such vessels, each with one flat or semi-curved face in proximity to a magnet face. Thus looking down from the top one (as depicted in FIG. 4) it would be clear the quadrupole could be divided so as to accommodate four separate triangle-like shaped vessels where the angle of each triangular vessel nearest the center of the magnetic device would be close to 90°. Since multipole devices can be constructed so as to create quite uniform radial gradients, cells would be well distributed and monolayered on such surfaces. As already noted, in order to place the sample in the most advantageous part of the magnetic gradients created by such multipole devices, vessels could be constructed such that some portion of the triangular structure, i.e. that part at the center of the quadrupole or multipole device could be lopped off (see shaded square region of FIG. 4). That modification would result in a vessel whose cross section is described as a truncated triangle or as referred to here as a truncated triangular vessel [TTV]. In such cases, the sample is not only placed in a region of the vessel that avoids the low gradient region but it also creates a situation where the travel distance a cell or some other entity required for capture is shortened—a significant advantage. These disclosures create two advantages: more rapid separations and separations of entities that might not be captured in conventional tubes because of the low gradient regions so described. These are significant advantages for creating more efficient separations.

There is another very significant advantage of the TTV so described, viz., the surface of the vessel whereupon collection takes place can either be an inserted microscope-like slide appropriately coated for binding to or catching elements that are magnetically collected or it can be a breakaway piece that can be treated as a microscope slide. In that way, magnetically labelled entities are magnetically translated onto a surface that can be removed, washed free of contaminating components without incurring the typical losses of transfers from one tube to some interrogation platform. Noteworthy is the fact that entities so bound to such a slide or slide like device can be removed from the magnetic gradient and be processed in the absence of a magnetic gradient. Not only does this feature facilitate processing and automation, it allows for washing off contaminants employing washes where the shearing forces can be carefully adjusted. That ability should be significant in discriminating between specific and non-specific binding, the latter presumably of lesser energy.

The following definitions are provided to facilitate an understanding of the present invention.

The term “target bioentities” as used herein refers to a wide variety of materials of biological or medical interest. Examples include hormones, proteins, peptides, lectins, oligonucleotides, drugs, chemical substances, nucleic acid molecules, (e.g., RNA and/or DNA) and particulate analytes of biological origin, which include bioparticles such as cells, viruses, bacteria and the like. In a preferred embodiment of the invention, rare cells, such as fetal cells in maternal circulation, or circulating cancer cells (CTC) may be efficiently isolated from non-target cells and/or other bioentities, using the compositions, methods and kits of the present invention.

The term “biological specimen” includes, without limitation, cell-containing bodily, fluids, peripheral blood, tissue homogenates, nipple aspirates, and any other source of rare cells that is obtainable from a human subject.

An exemplary tissue homogenate may be obtained from the sentinel node in a breast cancer patient.

The term “determinant”, when used in reference to any of the foregoing target bioentities, may be specifically bound by a biospecific ligand or a biospecific reagent, and refers to that portion of the target bioentity involved in, and responsible for, selective binding to a specific binding substance, the presence of which is required for selective binding to occur. In fundamental terms, determinants are molecular contact regions on target bioentities that are recognized by receptors in specific binding pair reactions.

The term “specific binding pair” as used herein includes antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin and virus-receptor interactions. Various other determinant-specific binding substance combinations are contemplated for use in practicing the methods of this invention, such as will be apparent to those skilled in the art.

The phrase “subcellular component” refers to intra and extra cellular molecules that are altered as a cell progresses from a normal to malignant phenotype. Assessment of altered expression levels or molecular structure of tumor associated molecules provides the clinician with valuable information to aid in the design of treatment and monitoring strategies.

The term “antibody” as used herein, includes immunoglobulins, monoclonal or polyclonal antibodies, immunoreactive immunoglobulin fragments, and single chain antibodies. Also contemplated for use in the invention are peptides, oligonucleotides or a combination thereof which specifically recognize determinants with specificity similar to traditionally generated antibodies.

The term “detectably label” is used to herein to refer to any substance whose detection or measurement, either directly or indirectly, by physical or chemical means, is indicative of the presence of the target bioentity in the test sample. Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecules.

As used herein, the phrase “high purity” means that preparation is at least 70%, preferably 80-90%, and more preferably between 95-98% purity for the cell or molecule of interest being isolated.

The phrase “to the substantial exclusion of” refers to the specificity of the binding reaction between the biospecific ligand or biospecific reagent and its corresponding target determinant. Biospecific ligands and reagents have specific binding activity for their target determinant yet may also exhibit a low level of non-specific binding to other sample components.

The term “early stage cancer” as used herein refers to those cancers which have been clinically determined to be organ-confined. Also included are tumors too small to be detected by conventional methods such as mammography for breast cancer patients, or X-rays for lung cancer patients. While mammography can detect tumors having approximately 2×10⁸ cells, the methods of the present invention should enable detection of circulating cancer cells from tumors approximating this size or smaller.

The term “enrichment” as used herein refers to the enrichment of mononuclear cells from a biological sample. In cases where peripheral blood is used as the starting materials, red cells are not counted when assessing the extent of enrichment. Using the method of the present invention, circulating epithelial cells may be enriched relative to leucocytes to the extent of at least 2,500 fold, more preferably 5,000 fold and most preferably 10,000 fold.

The following example is provided to facilitate practice of the present invention. It is not intended to limit the invention in any way.

### EXAMPLE I

It is well recognized that quadrupole, hexapole and other multipole magnetic separators are capable of producing radial gradient or gradients that are quite uniform [U.S. Pat. Nos. 5,466,574, 5,795,470]. Hence, in one case a test tube containing, for example, a ferrofluid when placed in such devices can be made to collect uniformly on the inner surfaces of the tube. If the quantity of ferrofluid is limited the uniform layer can be a monolayer. This concept was employed very effectively for immunoassays, (See U.S. Pat. No. 5,660,990) where a monolayer of ferrofluid to which was attached the component parts of sandwich immunoassays could be created. This concept in that case was used advantageously, eliminating and or simplifying processing steps. Thus, washing away contaminants that would normally be trapped in magnetic collection or NSB components can be accomplished by literally ‘hosing down the surface’ as opposed to repeated resuspensions and collections. In examples cited therein, ELISA or chemi-luminescent ‘read out’ reactions could be done without resuspension. In the case of cells, and particularly with fragile cells as is the case with CTV, not having to re-suspend is especially advantageous.

In the case of cell separations, the utility of creating monolayers of cells on the inside walls of tubes is generally not done [excepting possibly for CTC enrichment where there are so few cells] primarily because there is usually not sufficient surface area for creation of a monolayer. For example, immuno-magnetic cell separations are typically done at concentrations of 2×10⁷ cells/ml and such separations are most often done in 12×75 mm test tubes. As such tubes have inner diameters of 1.0 cm; a reasonable approximation is that the inner surface area, i.e. the collection surface, of such a tube filled to 1.0 ml (an area of 3.14 cm²) can accommodate about 4.7×10⁶ cells [based on calculations of the numbers of cells that can be monolayered per 1.0 cm² which is somewhere between 1.3 and 1.9×10⁶ cells]. That means that if a subpopulation of more than about 30% is targeted then collected cells begin building second layers when about 20% of the cells are collected. In the case of a depletion of, for example, CD45 depletion from PBMC separated at a concentration of 2×10⁷ cells/ml there will be more than four layers of cells on the collection wall. It would seem reasonable to suggest that as the number of cell layers increases, the retention of collected cells, particularly on the outermost layers would be in peril simply by trying to remove unbound or untargeted cells. The role that might play in achieving high log depletions of CD45 cells where highly effective depletions would not be insignificant. Additionally, in the case where the enrichment or isolation of some rare cell species that might occur from PBMC preps prepared from blood or some other bodily fluid, such as might be the case of CTC, where the approach is to deplete every cell except CTC, the large scale depletion of all cells excepting CTC can well be problematic because of entrapment of the desired cells within the layers of the depleted cells. Such approaches benefit significantly by monolayering the depleted cells. It is even more beneficial to not only monolayer them but to also ‘catch’ them by means disclosed herein.

There are many protocols as well as a variety of useful binding pair reactions available for construction of ferrofluid or particle conjugates that at least bi-specific such that they will bind to at least two unrelated surfaces that are useful in this invention. For example, if the goal is to catch all CD45 cells from PBMC, a ferrofluid conjugated to anti CD45 as well as streptavidin will suffice. Further a simple means for constructing such a conjugate would be to first synthesize a streptavidin ferrofluid which is then brought into contact with a limited amount of mono-biotin CD45 Mab so as to leave sufficient numbers of streptavidin free to bind biotin that would be placed on the vessel wall or in the case of a slide containing collection surface of a specially designed vessel. Thus cells would be incubated with this ferrofluid conjugate which would lead to targeted cells being magnetically labelled. That would be followed by magnetic separation onto a surface that has biotin or some biotin derivative covalently attached. The chemistries for attaching a spectrum of molecules to glass surfaces are well known to those skilled in the art [Weetall, Applied Biochemistry and Biotechnology 1993, 41, 157-188.; Meilczarski et al, Journal of Colloid and Interface Science 2011, 362, 532-539].

Alternatively, via an indirect method a poly biotinylated Mab having some 3-9 biotins/Mab could be employed, or any other biotinylated cell specific agent, which when incubated with a cell mixture will label target cells. Depending on the affinity of the specific agent used and the degree of labelling required to achieve magnetic capture, excess unbound agent may have to be removed, in which case centrifugation or even microfluidic sized based devices could be employed. Next a streptavidin conjugated ferrofluid would be added, incubated and separated magnetically onto an appropriate surface. Where the desired results are ‘catch’ only, a collection surface bearing biotin or some biotin conjugate will not only suffice but will produce strong bonding to this solid support. In the case where ‘catch and release’ is the desired outcome, it would be a simple matter to attach des-biotin to the collecting surface as it binds significantly less avid to streptavidin and its reaction with streptavidin is readily reversed by the addition of biotin, thus resulting in release.

There are a variety of member pair reactions that can be utilized for non-covalently attaching an entity to a surface such as glass or plastic/polymers. They include antibody-haptene reactions where the antibody could either be immobilized on the collecting surface or on the magnetic nano-particle. Antibodies to isotypes along with the isotype could be used, or an anti-isotype that will bind the Mab used in the targeting reaction could be employed by immobilizing it to the collecting surface. A simple anti-Fc immobilized to a collecting surface will similarly ‘catch’ targets that are labelled with target Mab. Likewise, protein A or G on the collecting surface would not only work but confer some advantages such as release upon small adjustment of pH. Many mammalian cells when cooled sufficiently with stand pH lowering very effectively.

There could be instances where there is concern that during incubation with nano-particles that their binding to the collection surface before binding to target could be disadvantageous. That is easily addressed by effectively ‘hiding’ the immobilized component behind some wall or barrier that can either prevent or delay that reaction from occurring. One simple solution is to coat collection surfaces with some agent that creates a barrier that can be eliminated. That can be accomplished by coating onto the collection surface a thin layer of gelatin or gelatin-like materials. Thus incubation is performed in a vessel so coated and that reaction is done below the melt temperature of the coating agent—a method advantageous for many separations as there are significant reasons to keep such reactions cool. Once incubation is complete, simply bringing up the surface temperature of the collection area to above the melt temperature of gelatin (near 37° C.) or some material having similar properties with even lower melt points will result in exposure of the ‘catch’ component. The technology for extraordinarily thin coating of gelatin and gelatin like materials is well known.

There are many processing and results advantages to COCAR. For example, whether cells are on a slide like surface that might have been part of a specifically designed vessel or on the inner walls of some radially symmetric vessel, it should be clear that the washing shear forces that can be applied can be effectively modulated and generally be greater than those where cells are held only by magnetic forces. Further, since entities so immobilized are held in place by multiple strong non-covalent bond there is no need for washing steps to be done in the presence of a magnetic field. Not only does this allow careful monitoring of wash shear forces applied in removing NSB components, it allows for visual inspection of the process—a very significant advantage. What also should be clear is that when done by properly depositing magnetically tagged entities directly onto a collection surface which is part of the vessel and when in particular that part can be used for interrogation with subsequent tools, there probability of cell or entity loss is nearly zero. For rare cell events that is very important. For obtaining high purity target cells, high log depletions of the methods disclosed here will be found to be very useful.

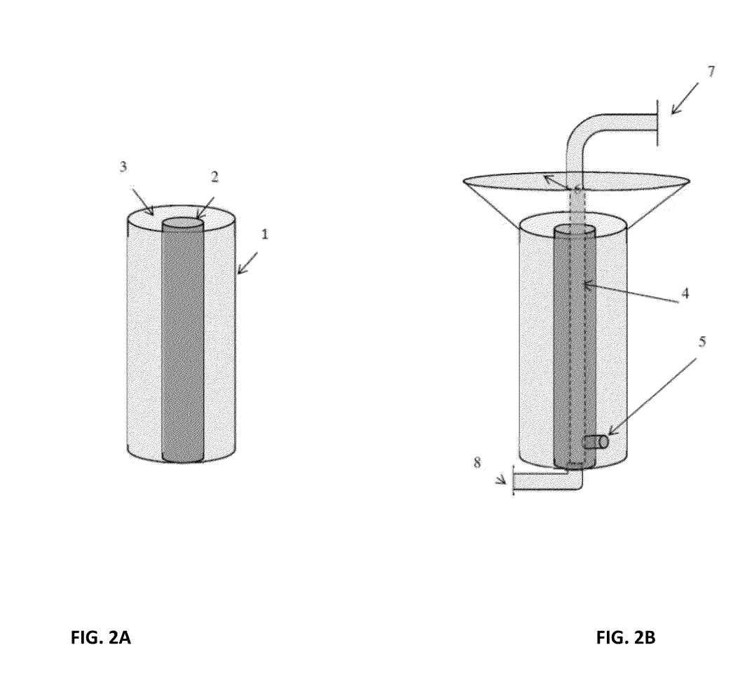

In order to better utilize the foregoing disclosures, we further disclose a family of separation vessels that take advantage of principles inherent in these disclosures. Parenthetically, there is no reason why the application of concepts disclosed cannot be used with existing vessels; however, it will be shown that the following disclosure vessel concepts markedly improve performance. FIG. 2a depicts the cross section of a tube like, in this case radially symmetric, vessel 1 that contains a post 2 at its center which creates an annular space 3 where sample to be separated is introduced. As already noted the creation of an annular space confers three significant benefits. First, the distance that a targeted cell or some other entity must travel to be collected on the outer wall of the annular space can be made significantly less than would be the case with no central post and that distance can be adjusted by adjusting the diameter of the central post. Secondly, by appropriate choices of the post diameter, the sample to be separated avoids the region of low to near zero magnetic gradients which exist for multipole magnetic separators such as quadrupole, hexapole, etc.; an effect which in concert with the shortened separation travel distance results in quicker separation. In addition to those positive benefits, the insertion of a post, depending on its diameter, can significantly increase the area upon which separation takes place. Table I. shows calculated results that illustrate this principle.

TABLE I
The relationship between post diameter, surface area of collection
and numbers of cells that can be monolayered for a 1.0 ml volume
in an annular space with 10 mm outer diameter.

| | Annular | column height | Surface | |
|---|---|---|---|---|
| Post Diameter | radius | per 1.0 ml | area | #cells/surface^{a} |
| 9 (mm) | 1 (mm) | 6.70 | 21.0 cm² | 31.5 × 10⁶ |
| 8 | 2 | 3.54 | 11.1 | 16.6 × 10⁶ |
| 7 | 3 | 2.49 | 7.8 | 11.7 × 10⁶ |
| 6 | 4 | 1.99 | 6.2 | 9.3 × 10⁶ |
| No post | | 1.27 | 4.0 | 6 × 10⁶ |

Footnote:
^{a}these numbers are calculated on the basis that a 1.0 cm² area can accommodate a monolayer of about 1.5 × 10⁶ leukocytes.

From Table I, it is reasonable to conclude that for cell separations done at 2×10⁷ cells/ml that a 10 mm I.D. tube can only collect about 30% of the cells in a positive selection in order for those cells to be monolayered. Consequently, a negative depletion of more than 30% creates a situation where cells begin to collect in additional layers. For a 2-3 log depletion, it would be necessary to use a tube with an inner post of 8 mm diameter in order for those cells to be monolayered.

As would be obvious to those skilled in the art, separation in an annular space of a few mm would take place very rapidly. On the other hand, loading, emptying and washing the surface of such narrow spaces would be difficult. To facilitate those operations, FIG. 2b depicts a vessel containing an annular space [VAS] where the central post is not solid but has in fact at least one channel 4 through which sample or other solutions can be introduced or removed from the annular space via the port 5 that connects the annular space to the channel 4. As would be obvious to someone skilled in the art, a precise slug of sample could be placed precisely within the annulus by literally pushing it with some denser media. Note also that the top of the vessel 6 is flared out such that liquid, e.g. wash solutions, can be introduced from the top of the vessel and removed via the inlet/outlet ports 7, 8. In this way, washing of magnetically bound materials on the annular surface closest to the magnets can be facilitated. With appropriate valves and given that liquids can be introduced either from the top or bottom of the annulus, it would be clear that a variety of operations can be performed within the VAS such as back and forth flow that would create an effective surface wash of separated components. Of course, with immuno-magnetically targeted component also non-covalently attached on the annular wall, the entire vessel can be removed from the magnetic field for such processing steps. There are multiple advantages to being able to perform reactions on entities, particularly cells, so immobilized because a reagent can be passed over such entities and unreacted reagent simply washed away as for example: immunochemical reactions, enzymatic reactions and the like.

To perform a ‘catch and release’ and harvest targeted cells in a small volume, the following principles could generally be followed noting that a VAS would be used where the outer wall of the annular space has coupled or adsorbed to it some member of a binding pair whose interaction with each other can readily be reversed. Such an operation might be done as follows: (I) to a mixture of cells targeting Mab that would have conjugated to it some member of a binding pair, such as biotin or desbiotin, is added, incubated for an appropriate interval and excess Mab removed by well-known centrifugation, microfluidic or filtration techniques (alternatively, by using Mabs of extremely high affinity, e.g. Kd in the picomolar range, there would be so little free Mab that many separations could be done without removing excess or unbound Mab; (II) to the mixture containing Mab labelled cells, a streptavidin ferrofluid would be added in an appropriate quantity, incubated either outside or within a VAS. In either case the VAS would be filled via the port 5 of FIG. 2b. To ensure placing the sample within the annulus a dense fluid would be employed to push and keep the sample in place (there are a variety of devices for controlled input of liquids) and after magnetic separation the VAS would be emptied of non-targeted components and washed by introducing wash buffers from the top 6 (or it can be done in reverse). It would be a simple matter to flow wash buffer in an oscillating fashion to create a ‘scrubbing’ action. (III) Once the collection surface of the VAS has been adequately cleaned of non-specifically bound components, the VAS can be removed from the magnetic gradient, if desired, and next a small quantity of elution buffer can be introduced and passaged over the collecting surface so as to elute cells as it passes.

One example of such an operation might be: if it is desired to perform a separation of a 4 ml sample and a VAS with an annulus of 3 mm is chosen, then referring to Table I the column length of the annulus needs to be about 10 cm. If after all the wash steps have been performed a quantity of elution buffer is added through the ‘plumbing’ that depicted in FIG. 2b, it would be a simple matter to introduce a plug of elution buffer in an amount of 100 or 200 ul. That plug could be pushed up into the annular space employing a ‘pushing’ denser liquid (e.g. ficoll, concentrated BSA or some non-interfering dense liquid.) and would create an annular ring, the height of which being either 2.5 or 5.0 mm. By passaging that elution ring over the collection surface, cells would be released into it and that volume could be recovered readily. For this example, the vessel wall could have attached to it des-biotin in some suitable form, cells adhered as described would be eluted with a biotin solution as biotin readily reverses the streptavidin-desbiotin reaction. In the event labelling Mab has on it desbiotin rather than biotin, the eluted cells would be free of magnetic material. In that case, it would be desirable to perform the elution with the VAS in a magnetic gradient so as to retain the ferrofluid and produce cells free of magnetic material.

In the procedure just described, there would be some reaction of conjugated ferrofluid with the vessel wall while at the same time that same ferrofluid is binding to biotin Mab labeled cells. There are two ways to avoid that situation in the event it proves negative. One is to perform the ferrofluid incubation outside the VAS. Alternatively, the collection surface could be treated so as to ‘hide’ the wall bound capture agent, i.e. biotin, des-biotin or some member of a binding pair. It would not be difficult to do that. One way to accomplish that is to coat the collection surface with a barrio such as gelatin that could be collapsed with gentle heating of the vessel just prior to separation. Technology for micro coating of gelatin or other similar materials is well known.

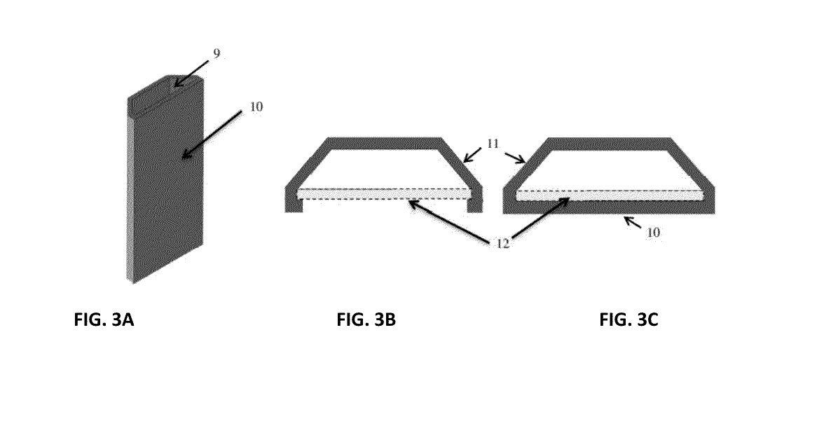

Another type of vessel that takes advantage of the principles of COCAR is one where targeted cells are magnetically manipulated directly onto a surface such as a microscope slide that can subsequently prepared for analysis and that can include all immunochemical, pathological and genetic testing. FIG. 3a depicts a ¾ view and a cross section (FIGS. 3b and 3c) of one such vessel. The vessel is open at the top 9 and has a structure and an internal volume that could be made suitable for performing various operations, e.g. mixing reagents, centrifugation, magnetic separation or any operations that vessels normally can be used for, on cells or other entities that could be candidates for immuno-magnetic separations. The vertical front wall of the vessel 10 approximates in one embodiment the dimensions of a microscope slide. That wall can, in fact, be a break away portion of the vessel that could indeed function as a slide for doing microscopy and all types of interrogations than can normally be done on cells. Alternatively, it would be a simple matter to construct a vessel with no front wall wherein a microscope slide or some similar surface could be slide into place such that sample could be placed therein without leakage. A cross section of this concept is shown in FIG. 3b. The dashed lines represent a slide cross section. Additionally, a vessel with a very thin front wall 10 could be constructed wherein slide like surfaces could be inserted behind that face, FIG. 3c. Note that the side walls 11 and 11′ of the vessel not square and in fact if they are extended they will meet and create a triangle with a base that is face 10. The purpose and function of this particular vessel is that to effect magnetic separation of its contents, side or face 10 is placed adjacent to a magnet surface such as in a quadrupole (or in the case of some other magnetic device placed where the gradient highest) where the pole face of the magnets employed are slightly larger than surface 10. Note also that four such vessels could be placed within one quadrupole (see FIG. 4). In fact, the ‘imaginary’ apex of the triangle formed by extending the tilted sides until they meet, in this case would be at the approximate center of the quadrupole. In that manner, magnetically labelled entities within the vessel will be pulled onto the inner surface of 10 in some radial fashion and when done under appropriate conditions will monolayer on that surface. Since the field of quadrupoles, or even appropriately constructed tripoles are radial, the side walls of the vessel, 11 and 11′ prime′ must be on radial lines or of larger angles so as to prevent entities that are being transported to surface 10 from hitting the sides of the vessel. These concepts are depicted in FIG. 4 which shows a cross section of four such vessels placed within a quadrupole device. Also shown in FIG. 4 are dashed lines extended from the sides of this vessel to the center of the quadrupole. In order to lessen the travel distance of magnetic entities to the collection surface and to also avoid regions of the quadrupole or some similar device where the magnetic gradients are effectively zero that portion of the vessel, were it triangular shaped, has been lopped off (the shaded regions within the square at the center of the quadrupole). Accordingly, the vessel is referred to as a truncated triangular vessel [TTV].

To perform an experiment where magnetically labelled cells are separated to collection side 10 of a TTV, caught on the surface by specific non-covalent bonding, subsequently cleansed of non-specifically bound components and then interrogated by means for analyzing and assessing cells which are now routine the following general procedure could be followed: (1) a slide having conjugated to one of its surfaces one member of a binding pair such as biotin or biotin as some part of some other entity ideally covalently linked to the slide, is inserted into a TTV that is capable of accepting it. If it is desirable to prevent the wall bound biotin from prematurely binding to any component in solution before separation initiated binding occurs, the slide could be coated with some barrier component such as gelatin. For simplicity a sample that has been incubated with Mab-biotin after which excess antibody has been removed by well-known methods and where that has been followed by incubation with a streptavidin ferrofluid, is introduced into a TTV and that is then positioned in a magnetic gradient such that gradient forces draw labelled materials to the slide surface. If a barrier is employed some step such as heating to melt or eliminate the barrior would need to be performed. After separation and non-covalent binding of magnetic entities, the slide or breakaway collecting surface would be removed, non-specifically bound materials removed by appropriate wash steps and subsequently analysis performed on platforms that can accept slides or similar surfaces with immobilized cells. It should be clear from the above disclosures on the TTV and the notion of direct depositing magnetic entities on surfaces for subsequent manipulation and observation that the surface on which such entities are deposited need not be flat or rigid as might generally be the case for the TTV. For example, for a VAS which might be generally radially symmetrical, an ultrathin piece of glass or plastic could be inserted such that it lies against the outer wall of the annulus therein. In that case, cells or appropriately labelled magnetic entities could be non-covalently attached to such sheets and subsequently withdrawn for processing and analysis.

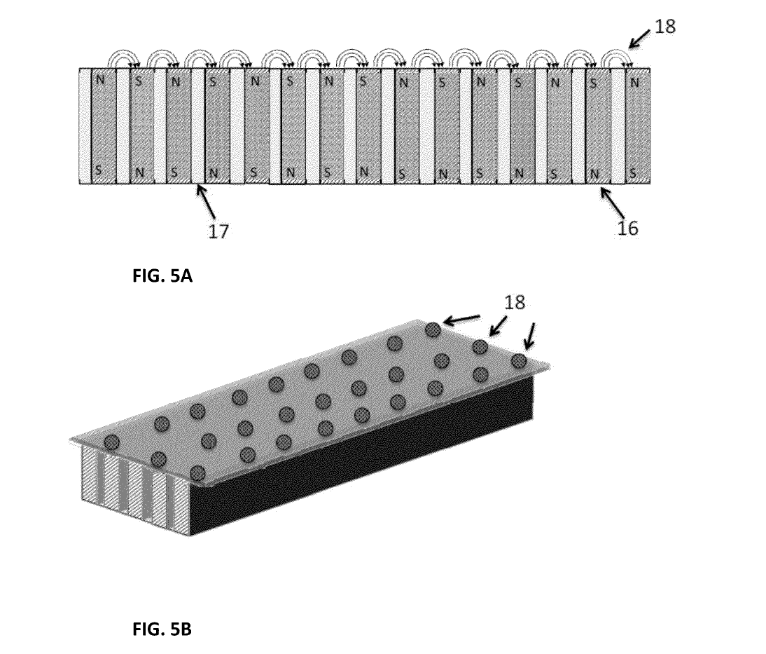

It should be noted that there are a variety of magnetic gradient forming devices that could be used with this invention. Already mentioned is the potential use of appropriately designed tri-pole magnet devices that would draw magnetic entities to the face of the central pole in some uniform fashion. Use of such a magnetic device does facilitate automation as TTV's or some similar vessel can easily be position for separation and removed for subsequent steps. Another simple magnetic arrangement can be constructed by placement of very narrow rectangular magnets side by side as shown in FIG. 5. Rectangular magnets, for example, 10 cm in length, 1.3 cm in height and 0.5 mm in width magnetized through the 1.3 cm dimension when stacked side by side with alternating polarity and with narrow non-magnetic rectangles placed between adjacent magnets create gradient ‘lines’ that pull cells and magnetic entities in general onto collecting surfaces of TTV in straight lines. FIG. 5b shows how a slide might look following the kind of separation described in FIG. 1. Clearly very sharp and thin ‘collection lines of cells’ would be obtained with magnets that are very narrow. Also for this array, very thin microscope like slides would be advantageous because gradients produced are strongest closest to the magnet faces. Such aligned collections of entities to be interrogated are useful in simplifying such analysis as once indexed an analyzer need not hunt for components of interest.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for isolating cells of interest, comprising;
a) obtaining a biological sample containing said cells of interest using a large gauge needle, thereby minimizing shear forces on said cells,
b) concentrating said cells in a solution in the presence of cell integrity preserving inhibitors,
c) contacting said cells with an antibody immuno-specific for an antigen on said cells of interest, said antibody being conjugated to a ferrofluid;
d) subjecting said cells to conditions suitable for an immunocomplex to form between said cells and said antibody conjugated to said ferrofluid;
e) separating immunocomplexed cells by applying a magnetic gradient to said solution, thereby isolating said cells of interest.

2. The method of claim 1, wherein said ferrofluid is conjugated to at least two antibodies or other binding moieties.

3. An apparatus for performing the method of claim 1, shown in FIG. 3.

4. An apparatus for performing the method of claim 1, shown in FIG. 4.

5. An apparatus for performing the method of claim 1, shown in FIG. 5.
